(19) United States
(12) Patent Application Publication
(10) Pub. No.: US 2021/0068439 A1
(43) Pub. Date: Mar. 11, 2021

(54) WELLBEING SUPPLEMENT FOR POSTPARTUM MATERNAL NUTRITION

(71) Applicant: MJN U.S. Holdings LLC, Evansville, IN (US)

(72) Inventor: Shireen Suresh DOULTANI, Evansville, IN (US)

(73) Assignee: MJN U.S. Holdings LLC, Evansville, IN (US)

(21) Appl. No.: 17/053,324

(22) PCT Filed: May 9, 2019
(86) PCT No.: PCT/EP2019/061979

§ 371 Date: Nov. 5, 2020

(30) Foreign Application Priority Data
May 9, 2018 (GB) 1807540.8

(51) Int. Cl. A23L 33/00; A61K 9/00; A61K 31/702; A61K 31/202; A61K 36/48; A61K 36/9066; A61K 33/06; A61K 31/59; A61K 35/747; A23L 33/125; A23L 33/12; A23L 33/135; A23L 33/155; A23L 33/105; A23L 33/16; A23C 9/13; A23C 9/133; A23C 9/20; A23C 11/10

(52) U.S. Cl. CPC A23L 33/40; A23Y 2220/73; A61K 31/702; A61K 31/202; A61K 36/48; A61K 36/9066; A61K 33/06; A61K 31/59; A61K 35/747; A23L 33/125; A23L 33/12; A23L 33/135; A23L 33/155; A23L 33/105; A23L 33/16; A23C 9/1315; A23C 9/1322; A23C 9/133; A23C 9/1307; A23C 9/203; A23C 11/106; A23V 2002/00; A61K 9/0056

(57) ABSTRACT

The present disclosure relates generally to methods and nutritional compositions for promoting or maintaining postpartum maternal health. The nutritional composition can include sialyllactose, a source of long chain polyunsaturated fatty acids (“LCPUFAs”), a probiotic, such as Lactobacillus rhamnosus GG (LGG), a nut extract, turmeric, fenugreek calcium and/or vitamin D. The nutritional composition is suitable for administration to postpartum mothers. Additionally, the disclosure provides methods for preventing or reducing the severity of postpartum depression and/or anxiety. The nutritional composition(s) provided herein can comprise various combinations of ingredients that provide additive and or/synergistic beneficial health effects to postpartum mothers.

### TECHNICAL FIELD

The present disclosure relates generally to methods and nutritional compositions for promoting or maintaining postpartum maternal health. The nutritional composition can include sialyllactose (e.g., 3′Sialyllactose and 6′Sialyllactose), a source of long chain polyunsaturated fatty acids (“LCPUFAs”), a probiotic, such as Lactobacillus rhamnosus GG (LGG), a nut extract, turmeric, fenugreek calcium and/or vitamin D. The nutritional composition is suitable for administration to postpartum mothers. Additionally, the disclosure provides methods for preventing or reducing the severity of postpartum depression and/or anxiety. The nutritional composition(s) provided herein can comprise various combinations of ingredients that provide additive and or/synergistic beneficial health effects to postpartum mothers.

### BACKGROUND ART

The demands on a mother following the birth of an infant are extensive. New mothers face many challenges including healing from the birth, drastic fluctuations in hormone levels, frequent breastfeeding and lack of sleep. It can be difficult for a mother to maintain her own health in the postpartum period, and in particular to consume the nutrients necessary to meet the nutritional demands of breastfeeding. Further, about 1 in 7 women experiences postpartum depression and/or anxiety.

Accordingly, there is a need in the art for a nutritional supplement that new mothers can consume on the go or while multitasking, which supports the mother's wellbeing with little effort on her part.

### BRIEF SUMMARY

Described herein are methods and nutritional compositions for promoting or maintaining postpartum maternal health. The nutritional composition can include sialyllactose (e.g., 3′Sialyllactose and 6′Sialyllactose), a source of long chain polyunsaturated fatty acids (“LCPUFAs”), a probiotic, such as Lactobacillus rhamnosus GG (LGG), a nut extract, turmeric, fenugreek calcium and/or vitamin D. The nutritional composition is suitable for administration to postpartum mothers to increase or maintain the health of the mother.

Additionally, the disclosure provides methods for preventing or reducing the severity of postpartum depression and/or anxiety. The nutritional composition(s) provided herein can comprise various combinations of ingredients that provide additive and or/synergistic beneficial health effects to postpartum mothers. In particular, the use of siallylactose and/or DHA may prevent or reduce the severity of postpartum depression and/or anxiety in a postpartum mother.

In one aspect, the disclosure relates to a nutritional composition for supporting the health of a postpartum mother, comprising siallylactose, a long chain polyunsaturated fatty acid (LCPUFA) and a probiotic. In certain embodiments, the nutritional composition also includes one or more of fenugreek, turmeric, a nut extract, calcium, vitamin D. For example, for a postpartum mother who is breastfeeding, a nutritional composition can include siallylactose, a LCPUFA, a probiotic, fenugreek, turmeric and/or other spices , nuts or a nut extract, calcium, and vitamin D. In other embodiments, for example, for a postpartum mother who is not breastfeeding, a nutritional composition can include sialyllactose, a LCPUFA, a probiotic, turmeric, calcium, and vitamin D.

In certain embodiments, the siallylactose is present in an amount of about 7 g to about 10 g per serving of the nutritional composition. In certain embodiments, the siallylactose includes 3′-Sialyllactose (3′-SL) and/or 6′-Sialyllactose (6′-SL).

In certain embodiments, LCPUFA is present in an amount of about 150 to about 200 mg per serving of the nutritional composition. For example, the LCPUFA can be present in an amount of about 50 mg to about 500 mg, about 100 mg to about 400 mg, about 150 mg to about 350 mg, or about 200 mg to about 300 mg. Suitable LCPUFAs include, but are not limited to, α-linoleic acid, γ-linoleic acid, linoleic acid, linolenic acid, docosahexaenoic acid (DHA), eicosapentaenoic acid (EPA) and arachidonic acid (ARA).

In certain embodiments, the LCPUFA includes docosahexaenoic acid (DHA). For example, the DHA can be present in an amount of about 50 mg to about 500 mg, about 100 mg to about 400 mg, about 150 mg to about 350 mg, or about 200 mg to about 300 mg.

In certain embodiments, the probiotic is present in an amount up to about 1.5×10¹⁰ cfu per serving of the nutritional composition. For example, the probiotic can be present in an amount between about 1.5×10⁹ and 1.5×10¹⁰. In certain embodiments, the probiotic includes Lactobacillus rhamnosus GG (LGG).

In certain embodiments, fenugreek is present in an amount up to about 1500 mg per serving of the nutritional composition.

In certain embodiments, turmeric is present in an amount sufficient to supply about 50 mg total curcuminoids per serving of the nutritional composition.

In certain embodiments, calcium is present in an amount of about 500 to about 1000 mg per serving of the nutritional composition. In certain embodiments, vitamin D is present in an amount of about 800 to about 1000 IU per serving of the nutritional composition.

In certain embodiments, the nutritional composition is in the form of a liquid, for example, a dairy-based drink, a fruit juice-based drink or a nut milk-based drink. In certain embodiments, the dairy-based drink is a yogurt drink. The liquid can be packaged as a 7-8 oz serving, for example. In other embodiments, the nutritional composition is in the form of a powder.

In another aspect, the disclosure relates to a method of supporting the health of a postpartum mother. The method includes providing to or administering to the mother any of the nutritional compositions described above.

In another aspect, the disclosure relates to a method of preventing or reducing the severity of postpartum depression and/or anxiety in a postpartum mother, the method comprising providing or administering to the mother any of the nutritional compositions described above.

It is to be understood that both the foregoing general description and the following detailed description present embodiments of the disclosure and are intended to provide an overview or framework for understanding the nature and character of the disclosure as it is claimed. The description serves to explain the principles and operations of the claimed subject matter. Other and further features and advantages of the present disclosure will be readily apparent to those skilled in the art upon a reading of the following disclosure.

### DETAILED DESCRIPTION

Reference now will be made in detail to the embodiments of the present disclosure, one or more examples of which are set forth hereinbelow. Each example is provided by way of explanation of the nutritional composition of the present disclosure and is not a limitation. In fact, it will be apparent to those skilled in the art that various modifications and variations can be made to the teachings of the present disclosure without departing from the scope of the disclosure. For instance, features illustrated or described as part of one embodiment, can be used with another embodiment to yield a still further embodiment.

Thus, it is intended that the present disclosure covers such modifications and variations as come within the scope of the appended claims and their equivalents. Other objects, features and aspects of the present disclosure are disclosed in or are apparent from the following detailed description. It is to be understood by one of ordinary skill in the art that the present discussion is a description of exemplary embodiments only and is not intended as limiting the broader aspects of the present disclosure.

The present disclosure relates generally to nutritional compositions and methods for supporting the health of a postpartum mother. In certain embodiments, the nutritional compositions described herein are used to provide nutrients to a postpartum mother. The disclosure also relates to nutritional compositions and methods for preventing or reducing the severity of postpartum depression and/or anxiety in a postpartum mother. In other embodiments, the disclosure relates to a method of making a nutritional composition as described herein, the method comprising combining one or more of the components described herein.

“Nutritional composition” means a substance or formulation that satisfies at least a portion of a postpartum mother's nutrient requirements. The terms “nutritional(s)”, “nutritional formula(s)”, “enteral nutritional(s)”, and “nutritional supplement(s)” are used as non-limiting examples of nutritional composition(s) throughout the present disclosure. Moreover, “nutritional composition(s)” may refer to liquids, powders, gels, pastes, solids, concentrates, suspensions, or ready-to-use forms of oral formulas (i.e., drinks).

“Nutritionally complete” means a composition that may be used as the sole source of nutrition, which would supply essentially all of the required daily amounts of vitamins, minerals, and/or trace elements in combination with proteins, carbohydrates, and lipids.

The nutritional composition of the present disclosure may be substantially free of any optional or selected ingredients described herein, provided that the remaining nutritional composition still contains all of the required ingredients or features described herein. In this context, and unless otherwise specified, the term “substantially free” means that the selected composition may contain less than a functional amount of the optional ingredient, typically less than 0.1% by weight, and also, including zero percent by weight of such optional or selected ingredient.

As applied to nutrients, the term “essential” refers to any nutrient that cannot be synthesized by the body in amounts sufficient to maintain health and that, therefore, must be supplied by the diet. The term “conditionally essential” as applied to nutrients means that the nutrient must be supplied by the diet under conditions when adequate amounts of the precursor compound is unavailable to the body for endogenous synthesis to occur.

“Postpartum mother” means a woman who has recently given birth to an infant (e.g., one or more infants). In certain embodiments, a postpartum mother is a mother who has given birth to an infant within the previous five years. In certain embodiments, a postpartum mother is a mother who has given birth within the previous three years. In certain embodiments, a postpartum mother is a mother who has given birth within the previous two years. In certain embodiments, a postpartum mother is a mother who has given birth within the previous year. In certain embodiments, a postpartum mother is a mother who has given birth within the previous 6 months. In certain embodiments, a postpartum mother is a mother who has given birth within the previous 3 months. In certain embodiments, a postpartum mother is a mother who has given birth within the previous 6 weeks. In certain embodiments, any mother who is breastfeeding can be considered a postpartum mother.

“Postpartum depression and/or anxiety” is a term understood in the art to mean a clinical depression occurring following the birth of an infant. Preventing or reducing the severity of postpartum depression means preventing or reducing the severity of one or more symptoms of postpartum depression including anger, anxiety, guilt, hopelessness, loss of interest or pleasure in activities, mood swings, panic attack, fatigue, loss of appetite, restlessness, depression, fear, repeatedly going over thoughts, crying, irritability, lack of concentration, unwanted thoughts, and insomnia.

“Probiotic” means a microorganism with low or no pathogenicity that exerts at least one beneficial effect on the health of the host. An example of a probiotic is LGG.

In an embodiment, the probiotic(s) may be viable or non-viable. As used herein, the term “viable”, refers to live microorganisms. The term “non-viable” or “non-viable probiotic” means non-living probiotic microorganisms, their cellular components and/or metabolites thereof. Such non-viable probiotics may have been heat-killed or otherwise inactivated, but they retain the ability to favorably influence the health of the host. The probiotics useful in the present disclosure may be naturally-occurring, synthetic or developed through the genetic manipulation of organisms, whether such source is now known or later developed.

The term “inactivated probiotic” means a probiotic wherein the metabolic activity or reproductive ability of the referenced probiotic organism has been reduced or destroyed. The “inactivated probiotic” does, however, still retain, at the cellular level, at least a portion its biological glycol-protein and DNA/RNA structure. As used herein, the term “inactivated” is synonymous with “non-viable”. More specifically, a non-limiting example of an inactivated probiotic is inactivated Lactobacillus rhamnosus GG (“LGG”) or “inactivated LGG”.

The term “cell equivalent” refers to the level of non-viable, non-replicating probiotics equivalent to an equal number of viable cells. The term “non-replicating” is to be understood as the amount of non-replicating microorganisms obtained from the same amount of replicating bacteria (cfu/g), including inactivated probiotics, fragments of DNA, cell wall or cytoplasmic compounds. In other words, the quantity of non-living, non-replicating organisms is expressed in terms of cfu as if all the microorganisms were alive, regardless whether they are dead, non-replicating, inactivated, fragmented etc.

All percentages, parts and ratios as used herein are by weight of the total composition, unless otherwise specified.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

As used herein, the term “about” should be construed to refer to both of the numbers specified as the endpoint(s) of any range. Any reference to a range should be considered as providing support for any subset within that range.

The amount of each ingredient of the nutritional compositions described herein is measured on a per serving basis. A serving can be from about 7 to about 8 oz and/or from about 1500 kcal to about 300 0 kcal

The nutritional compositions disclosed herein can contain sialyllactose. Suitable forms of sialyllactose for use in the compositions and method herein include 3′-Sialyllactose (3′-SL) and/or 6′-Sialyllactose (6′-SL).

Sialyllactose can be purified from a milk product, as described in U.S. Pat. No. 8,445,053. A milk product can be milk or any milk product derived from a ruminant or another milk producing animal. The milk product can for example be a bovine whey product, such as whey retentate or whey permeate. The milk product can also be the mother liquor from preparation of lactose from whey. It is also possible to use milk permeates, milk retentates, fractionated milk retentate or any other milk products containing sialyllactose. Sialyllactose can also be synthesized as described in U.S. Pat. Nos. 8,507,227 and 9,637,768. Sialyllactose can be obtained from a commercial source, such as Arla Foods Inc., Basking Ridge, N.J. or Jennewein Biotechnology GmbH, Rheinbreitbach, Germany.

In certain embodiments, the sialyllactose is present in an amount of about 7 g to about 10 g per serving of the nutritional composition. In certain embodiments, sialyllactose is present in an amount of about 8 g to about 10 g, about 8 g to about 9 g, about 9 g to about 10 g, about 7 g, about 8 g, about 9 g, or about 10 g.

In some embodiments the nutritional composition may also include a source of LCPUFAs. Non-limiting examples of LCPUFAs include, but are not limited to, DHA, ARA, linoleic (18:2 n-6), γ-linolenic (18:3 n-6), dihomo-γ-linolenic (20:3 n-6) acids in the n-6 pathway, α-linolenic (18:3 n-3), stearidonic (18:4 n-3), eicosatetraenoic (20:4 n-3), eicosapentaenoic (20:5 n-3), and docosapentaenoic (22:6 n-3). In one embodiment the amount of LCPUFA in the nutritional composition is at least about 150 mg per serving. The amount of LCPUFA may vary from about 150 mg per serving to about 200 mg per serving, from about 160 mg to about 190 mg per serving, or from about 170 mg to about 180 mg per serving.

In some embodiments, the LCPUFA included in the nutritional composition may comprise DHA. If included, the source of DHA and/or ARA may be any source known in the art such as marine oil, fish oil, single cell oil, egg yolk lipid, and brain lipid. In some embodiments, the DHA and ARA are sourced from single cell Martek oils, DHASCO® and ARASCO®, or variations thereof. The DHA and ARA can be in natural form, provided that the remainder of the LCPUFA source does not result in any substantial deleterious effect on the postpartum mother. Alternatively, the DHA and ARA can be used in refined form.

In an embodiment, sources of DHA and ARA are single cell oils as taught in U.S. Pat. Nos. 5,374,657; 5,550,156; and 5,397,591, the disclosures of which are incorporated herein in their entirety by reference. Nevertheless, the present disclosure is not limited to only such oils.

In one embodiment the amount of DHA in the nutritional composition is at least about 150 mg per serving. The amount of DHA may vary from about 150 mg to about 200 mg per serving, from about 160 mg to about 190 mg per serving, from about 170 mg to about 180 mg per serving, from about 150 mg to about 190 mg per serving, from about 150 mg to about 180 mg per serving, from about 150 mg to about 170 mg per serving, from about 160 mg to about 200 mg per serving, from about 170 mg to about 200 mg per serving, from about 180 mg to about 200 mg per serving.

In some embodiments, the probiotic of the nutritional composition comprises Lactobacillus rhamnosus GG (ATCC number 53103). In other embodiments, the disclosed nutritional composition(s) described herein may comprise a probiotic other than LGG, either in addition to LGG or in place of LGG. Additional probiotics that may be included in the nutritional composition include, but are not limited to: Bifidobacterium species, Bifidobacterium longum BB536 (BL999, ATCC: BAA-999), Bifidobacterium longum AH1206 (NCIMB: 41382), Bifidobacterium breve AH1205 (NCIMB: 41387), Bifidobacterium infantis 35624 (NCIMB: 41003), and Bifidobacterium animalis subsp. lactis BB-12 (DSM No. 10140) or any combination thereof.

In some embodiments, the nutritional composition includes a probiotic such as LGG in an amount up to about 1.5×10¹⁰ per serving. In other embodiments, the nutritional composition comprises a probiotic in an amount of from about 1×10⁶ cfu/100 kcal to about 1×10⁹ cfu/100 kcal. Still, in certain embodiments, the nutritional composition may include a probiotic in an amount of from about 1×10⁷ cfu/100 kcal to about 1×10⁸ cfu/100 kcal.

In certain embodiments, fenugreek is included in the composition. Fenugreek is particularly useful for inclusion in a nutritional composition for a postpartum mother who is breastfeeding, because it is known to stimulate milk production. Fenugreek can be added in an amount up to 1500 mg per serving of the composition. For example, fenugreek may be present in an amount of from about 0.1 mg to about 1500 mg, from about 1 mg to about 1500 mg, from about 1 mg to about 1000 mg, from about 10 mg to about 1500 mg, from about 10 mg to about 1000 mg, from about 50 mg to about 1500 mg, from about 50 mg to about 1000 mg, from about 100 mg to about 1500 mg or from about 100 mg to about 1000 mg.

In certain embodiments, turmeric is included in the composition. The anti-inflammatory properties of turmeric can be useful to a postpartum mother. Turmeric contains curcuminoid compounds such as curcumin, demethozycurcumin and bis-demethoxycurcumin. Turmeric can be added in an amount so as to provide up to about 50 mg total curcuminoids per serving of the composition. In certain embodiments, the nutritional composition comprises turmeric in an amount sufficient to about 0.1 to about 50 mg total curcuminoids per serving, about 1 to about 50 mg total curcuminoids per serving, about 5 to about 50 mg total curcuminoids per serving, or about 25 to about 50 mg curcuminoids per serving.

In certain embodiments, nuts or a nut extract is included in the composition. Recent evidence suggests that consumption of nuts at an early age may prevent nut allergies in infants. (See, Du Toit et al, (2015) “Randomized Trial of Peanut Consumption in Infants at Risk for Peanut Allergy,” N ENGLJ MED372:803-813.) Accordingly, inclusion of nuts or a nut extract in a nutritional composition for a postpartum mother breastfeeding her infant may be useful in preventing nut allergies in the infant. The nuts (e.g., nut powder) or nut extract can be included in an amount of 5-8 oz per serving of the nutritional composition. In certain embodiments, the nuts or nut extract is present in an amount of about 0.1 oz to about 5 oz, about 0.5 oz to about 5 oz, about 1 oz to about 5 oz, or about 3 oz to about 5 oz of the nutritional composition.

Exemplary nut extracts suitable for use in the present compositions include any extract containing an antigenic protein of a peanut or tree nut, including extracts in liquid or powder form. Exemplary tree nuts include cashews, pistachios, hazelnuts, walnuts, pecans, Brazil nuts, almonds and pine nuts.

In certain embodiments, calcium is included in the nutritional composition. The calcium can be added to the nutritional composition in the form of calcium citrate, calcium acetate, calcium gluconate, calcium chloride, calcium lactate, calcium phosphate, or calcium sulfate, etc. Calcium can be added in an amount up to 1000 mg per serving of the nutritional composition. For example, calcium can be added in an amount of about 500 mg to about 1000 mg per serving. In certain embodiments, calcium is added in an amount of 500 mg per serving of the nutritional composition. In other embodiments, calcium is added in an amount of 1000 mg per serving of the nutritional composition.

In certain embodiments, vitamin D can be included in the nutritional composition. The vitamin D can be added to the nutritional composition in the form of calciferol, cholecalciferol, vitamin D₃, and/or 1,25,-dihydroxyvitamin D. The vitamin D can be included, for example, as a stabilized vitamin D powder of vitamin D oil. In certain embodiments, vitamin D can be included in an amount of about 800 to about 1000 IU per serving of the nutritional composition. In certain embodiments, vitamin D is included in an amount of about 800 IU per serving of the nutritional composition. In certain embodiments, vitamin D is included in an amount of about 1000 IU per serving of the nutritional composition.

One or more additional vitamins and/or minerals may also be included in the nutritional composition in amounts sufficient to supply the daily nutritional requirements of a postpartum mother. For example, the composition may optionally include, but is not limited to, one or more of the following vitamins or derivations thereof: vitamin B₁ (thiamin, thiamin pyrophosphate, TPP, thiamin triphosphate, TTP, thiamin hydrochloride, thiamin mononitrate), vitamin B₂ (riboflavin, flavin mononucleotide, FMN, flavin adenine dinucleotide, FAD, lactoflavin, ovoflavin), vitamin B₃ (niacin, nicotinic acid, nicotinamide, niacinamide, nicotinamide adenine dinucleotide, NAD, nicotinic acid mononucleotide, NicMN, pyridine-3-carboxylic acid), vitamin B₃-precursor tryptophan, vitamin B₆ (pyridoxine, pyridoxal, pyridoxamine, pyridoxine hydrochloride), pantothenic acid (pantothenate, panthenol), folate (folic acid, folacin, pteroylglutamic acid), vitamin B₁₂ (cobalamin, methylcobalamin, deoxyadenosylcobalamin, cyanocobalamin, hydroxycobalamin, adenosylcobalamin), biotin, vitamin C (ascorbic acid), vitamin A (retinol, retinyl acetate, retinyl palmitate, retinyl esters with other long-chain fatty acids, retinal, retinoic acid, retinol esters), vitamin E (α-tocopherol, α-tocopherol acetate, α-tocopherol succinate, α-tocopherol nicotinate, α-tocopherol), vitamin K (vitamin K₁, phylloquinone, naphthoquinone, vitamin K₂, menaquinone-7, vitamin K₃, menaquinone-4, menadione, menaquinone-8, menaquinone-8H, menaquinone-9, menaquinone-9H, menaquinone-10, menaquinone-11, menaquinone-12, menaquinone-13), choline, inositol, β-carotene and any combinations thereof.

In certain embodiments, the composition may optionally include, but is not limited to, one or more of the following minerals or derivations thereof: boron, chloride, chromium, chromium chloride, chromium picolonate, copper, copper sulfate, copper gluconate, cupric sulfate, fluoride, iron, carbonyl iron, ferric iron, ferrous fumarate, ferric orthophosphate, iron trituration, polysaccharide iron, iodide, iodine, magnesium, magnesium carbonate, magnesium hydroxide, magnesium oxide, magnesium stearate, magnesium sulfate, manganese, molybdenum, phosphorus, potassium, potassium phosphate, potassium iodide, potassium chloride, potassium acetate, selenium, sulfur, sodium, docusate sodium, sodium chloride, sodium selenate, sodium molybdate, zinc, zinc oxide, zinc sulfate and mixtures thereof. Non-limiting exemplary derivatives of mineral compounds include salts, alkaline salts, esters and chelates of any mineral compound.

The minerals can be added to the nutritional compositions in the form of salts such as calcium phosphate, calcium glycerol phosphate, sodium citrate, potassium chloride, potassium phosphate, magnesium phosphate, ferrous sulfate, zinc sulfate, cupric sulfate, manganese sulfate, and sodium selenite.

The disclosed nutritional composition described herein, can, in some embodiments also comprise an effective amount of iron. The iron may comprise encapsulated iron forms, such as encapsulated ferrous fumarate or encapsulated ferrous sulfate or less reactive iron forms, such as ferric pyrophosphate or ferric orthophosphate.

The nutritional compositions of the present disclosure may optionally include one or more of the following flavoring agents, including, but not limited to, flavored extracts, volatile oils, cocoa or chocolate flavorings, peanut butter flavoring, cookie crumbs, vanilla or any commercially available flavoring. Examples of useful flavorings include, but are not limited to, pure anise extract, imitation banana extract, imitation cherry extract, chocolate extract, pure lemon extract, pure orange extract, pure peppermint extract, honey, imitation pineapple extract, imitation rum extract, imitation strawberry extract, or vanilla extract; or volatile oils, such as balm oil, bay oil, bergamot oil, cedarwood oil, cherry oil, cinnamon oil, clove oil, or peppermint oil; peanut butter, chocolate flavoring, vanilla cookie crumb, butterscotch, toffee, and mixtures thereof. The amounts of flavoring agent can vary greatly depending upon the flavoring agent used. The type and amount of flavoring agent can be selected as is known in the art.

The nutritional compositions of the present disclosure may optionally include one or more emulsifiers that may be added for stability of the final product. Examples of suitable emulsifiers include, but are not limited to, lecithin (e.g., from egg or soy), alpha lactalbumin and/or mono- and di-glycerides, and mixtures thereof. Other emulsifiers are readily apparent to the skilled artisan and selection of suitable emulsifier(s) will depend, in part, upon the formulation and final product.

The nutritional compositions of the present disclosure may optionally include one or more preservatives that may also be added to extend product shelf life. Suitable preservatives include, but are not limited to, potassium sorbate, sodium sorbate, potassium benzoate, sodium benzoate, calcium disodium EDTA, and mixtures thereof.

The nutritional compositions of the present disclosure may optionally include one or more stabilizers. Suitable stabilizers for use in practicing the nutritional composition of the present disclosure include, but are not limited to, gum arabic, gum ghatti, gum karaya, gum tragacanth, agar, furcellaran, guar gum, gellan gum, locust bean gum, pectin, low methoxyl pectin, gelatin, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, and mixtures thereof.

The nutritional compositions of the disclosure may provide minimal, partial or total nutritional support. The compositions may be nutritional supplements or meal replacements. The compositions may, but need not, be nutritionally complete. In an embodiment, the nutritional composition of the disclosure is nutritionally complete and contains suitable types and amounts of lipid, carbohydrate, protein, vitamins and minerals. The amount of lipid or fat included can be up to about 20 g per serving, typically varying between about 5.4 to about 20 g per serving. The amount of protein included can be up to about 6 g per serving, typically varying from about 3 g to about 6 g per serving. The amount of carbohydrate included can be up to about 30 g per serving, typically varying from about 11 g to about 30 g per serving.

In certain embodiments, the nutritional composition comprises no added sugar. In certain embodiments, a single serving of the nutritional composition contains 175-300 calories (kcal). The nutritional composition may be sweetened using artificial sweeteners (e.g., Stevia) or hydrolyzed lactose. In certain embodiments, an artificial sweetener is included in a fruit-based drink. In other embodiments, hydrolyzed lactose is included in a dairy-based drink.

The disclosed nutritional composition(s) may be provided in any form known in the art, such as a powder, a gel, a suspension, a paste, a solid, a liquid, a liquid concentrate, a reconstituteable powdered milk substitute or a ready-to-use product. Nutritional compositions of the present disclosure include, for example, orally-ingestible, health-promoting substances including, for example, foods, beverages, tablets, gummies, capsules and powders. Moreover, the nutritional composition of the present disclosure may be standardized to a specific caloric content, it may be provided as a ready-to-use product, or it may be provided in a concentrated form.

In a preferred embodiment, the nutritional composition is provided in a ready-to-use dairy-based drink, fruit-based drink, nut milk-based drink, or soy milk-based drink. In certain embodiments, the nutritional composition is provided in a yogurt drink such as a yogurt smoothie. In certain embodiments, the fruit-based drink comprises apple juice, orange juice, pineapple juice, mango juice, grape juice or any combination thereof. In certain embodiments the nut milk-based drink comprises peanut milk, cashew milk, pistachio milk, hazelnut milk, walnut milk, pecan milk, Brazil nut milk, almond milk and pine nut milk.

In some embodiments, the nutritional composition is in powder form with a particle size in the range of 5 μm to 1500 μm, more preferably in the range of 10 μm to 300 μm. The nutritional composition in powder form may be provided in a single-serving packet or pouch.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

### FORMULATION EXAMPLES

### Example 1

In this example, provided is a dairy-based supplement for all postpartum mothers (those breastfeeding and those not breastfeeding) provides sialyllactose, DHA, LGG, turmeric, calcium, and vitamin D. The supplement may be in the form of a powder, refrigerated extended shelf life liquid, gummy, or pudding.

### Example 1: Diary Based Drink

KEY INGREDIENT AMOUNT PER SERVING: SIALYLLACTOSE (FOR 10 GM PER SERVING)—(BOVINE SOURCE AT 4-4.2% SAL IN RAW MATERIAL)—240-300 GMS (OVERAGE MAY BE ADDED TO ACCOUNT FOR DEGRADATION AND/OR PROCESSING LOSSES AND/OR SHELF LIFE); SIALYLLACTOSE (FOR 10 GM PER SERVING)—(SYNTHETIC SOURCE AT 90% PURITY)—<=2 GM (OVERAGE MAY BE ADDED TO ACCOUNT FOR DEGRADATION AND/OR PROCESSING LOSSES AND/OR SHELF LIFE); FENUGREEK SEED POWDER (FOR 1500 MG PER SERVING)—UPTO 3 GMS; TURMERIC POWDER (FOR 50 MG OF CURCUMINOIDS PER SERVING)—<=2.4 GMS; DHA OIL (FOR 200 MG PER SERVING)—0.5 GMS (OVERAGE MAY BE ADDED TO ACCOUNT FOR DEGRADATION AND/OR PROCESSING LOSSES AND/OR SHELF LIFE); VITAMIN D CHOLECALCIFEROL CONCENTRATE LIQUID (FOR 1000 IU PER SERVING)—1 MG; CALCIUM CITRATE (FOR 1000 MG PER SERVING)—2.35-4.7 GMS (OVERAGE MAY BE ADDED TO ACCOUNT FOR DEGRADATION AND/OR PROCESSING LOSSES AND/OR SHELF LIFE); LGG (PER SERVING)—<=1 GM PURE CULTURE (OVERAGE MAY BE ADDED TO ACCOUNT FOR DEGRADATION AND/OR PROCESSING LOSSES AND/OR SHELF LIFE); YOGURT/MILK; WHEY PROTEIN CONCENTRATE; HYDROLYZED LACTOSE AND/OR ARTIFICIAL SWEETENERS; VITAMINS; MINERALS.

| | INGREDIENTS PER 8 OZ SERVING | |
|---|---|---|
| DAIRY BASED DRINK | AMOUNT | |
| LOW FAT MILK POWDER | 17 | GMS |
| WPC | 6 | GMS |
| DHA | 0.512 | GMS |
| HYDROLYZED LACTOSE | 8 | GMS |
| SAL (95% PURITY) | 7.7 | GMS |
| FENUGREEK POWDER | 3 | GMS |
| TURMERIC | 2.4 | GMS |
| VITAMIN D | 0.002 | GMS |
| CALCIUM CITRATE | 4.7 | GMS |
| OTHER (PROBIOTIC, STABILIZERS ETC) | 1 | GM |
| MILK FLAVOR | 0.05 | GMS |
| OTHER VITAMINS | 0.1 | GMS |
| WATER | 177 | GMS |
| TOTAL | 8 | OZ |
| CALORIES | 175-180 | |

| DAIRY BASED POWDER | PER 100 GMS POWDER | |
|---|---|---|
| LOW FAT MILK POWDER | 33.69 | GMS |
| WPC | 11.89 | GMS |
| DHA | 1.01 | GMS |
| HYDROLYZED LACTOSE | 15.85 | GMS |
| SAL (95% PURITY) | 15.26 | GMS |
| FENUGREEK POWDER | 5.94 | GMS |
| TURMERIC | 4.76 | GMS |
| VITAMIN D | 3.96 | MG |
| CALCIUM CITRATE | 9.31 | GMS |
| OTHER (PROBIOTIC, STABILIZERS ETC) | 1.98 | GM |
| MILK FLAVOR | 0.10 | GMS |
| OTHER VITAMINS | 0.20 | GMS |
| TOTAL | 100 | GMS |

### Example 2

In this example, a supplement for all postpartum mothers (those breastfeeding and those not breastfeeding) provides sialyllactose, DHA, LGG cultures, nuts or nut extract, turmeric, calcium, and vitamin D.

Dairy and Nut Based Product: Powder, Refregerated ESL Liquid or Gummy or Pudding

SIALYLLACTOSE (FOR 10 GM PER SERVING)—(BOVINE SOURCE AT 4-4.2% SAL IN RAW MATERIAL)—240-300 GMS (OVERAGE MAY BE ADDED TO ACCOUNT FOR DEGRADATION AND/OR PROCESSING LOSSES AND/OR SHELF LIFE); SIALYLLACTOSE (FOR 10 GM PER SERVING)—(SYNTHETIC SOURCE AT 90% PURITY)—<=2 GM (OVERAGE MAY BE ADDED TO ACCOUNT FOR DEGRADATION AND/OR PROCESSING LOSSES AND/OR SHELF LIFE); FENUGREEK SEED POWDER (FOR 1500 MG PER SERVING)—UPTO 3 GMS; TURMERIC POWDER (FOR 50 MG OF CURCUMINOIDS PER SERVING)—2.4 GMS (OVERAGE MAY BE ADDED TO ACCOUNT FOR DEGRADATION AND/OR PROCESSING LOSSES AND/OR SHELF LIFE); DHA OIL (FOR 200 MG PER SERVING)—0.5 GMS (OVERAGE MAY BE ADDED TO ACCOUNT FOR DEGRADATION AND/OR PROCESSING LOSSES AND/OR SHELF LIFE); VITAMIN D CHOLECALCIFEROL CONCENTRATE LIQUID (FOR 1000 IU PER SERVING)—1 MG (OVERAGE MAY BE ADDED TO ACCOUNT FOR DEGRADATION AND/OR PROCESSING LOSSES AND/OR SHELF LIFE); CALCIUM CITRATE (FOR 1000 MG PER SERVING)—2.35-4.7 GMS; NUT POWDER PER SERVING—UPTO 28 GMS; LGG (PER SERVING)—<=1 GM PURE CULTURE (OVERAGE MAY BE ADDED TO ACCOUNT FOR DEGRADATION AND/OR PROCESSING LOSSES AND/OR SHELF LIFE); YOGURT/MILK; HYDROLYZED LACTOSE; ARTIFICIAL SWEETENERS; VITAMINS; MINERALS.

| | INGREDIENTS PER 8 OZ SERVING | |
|---|---|---|
| DAIRY AND NUT BASED DRINK | AMOUNT | |
| MILK POWDER | 12.75 | GMS |
| NUT POWDER | 20.3 | GMS |
| DHA | 0.512 | GMS |
| HYDROLYZED LACTOSE | 10 | GMS |
| SAL (95% PURITY) | 7.7 | GMS |
| FENUGREEK POWDER | 3 | GMS |
| TURMERIC | 2.4 | GMS |
| VITAMIN D | 0.002 | GMS |
| CALCIUM CITRATE | 4.7 | GMS |
| OTHER (PROBIOTIC, STABILIZERS ETC) | 1 | GM |
| OTHER VITAMINS | 0.1 | GM |
| WATER | 185 | GMS |
| TOTAL | 8 | OZ |
| CALORIES | 275-300 | |

| | | |
|---|---|---|
| | AMOUNT PER 100 | AMOUNT PER 100 |
| DAIRY and NUT BASED POWDER | GMS POWDER | GMS POWDER |

| | | |
|---|---|---|
| MILK POWDER | 20.41 | GMS |
| NUT POWDER | 32.50 | GMS |
| DHA | 0.82 | GMS |
| HYDROLYZED LACTOSE | 16.01 | GMS |
| SAL (95% PURITY) | 12.33 | GMS |
| FENUGREEK POWDER | 4.80 | GMS |
| TURMERIC | 3.84 | GMS |
| VITAMIN D | 3.20 | MG |
| CALCIUM CITRATE | 7.52 | GMS |
| OTHER (PROBIOTIC, STABILIZERS ETC) | 1.60 | GM |
| OTHER VITAMINS | 0.16 | GM |
| SUM | 100 | GMS |

### Example 3

In this example, a supplement for breastfeeding postpartum mothers provides sialyllactose, DHA, LGG, fenugreek, nuts or nut extract, turmeric, calcium and Vitamin D.

Dairy, Nut and Fruit Based Product: Powder, Refrigerated ESL Liquid or Gummy or Pudding

The Ingredients are as follows: SIALYLLACTOSE (FOR 10 GM PER SERVING)—(BOVINE SOURCE AT 4-4.2% SAL IN RAW MATERIAL)—240-300 GMS (OVERAGE MAY BE ADDED TO ACCOUNT FOR DEGRADATION AND/OR PROCESSING LOSSES AND/OR SHELF LIFE); SIALYLLACTOSE (FOR 10 GM PER SERVING)—(SYNTHETIC SOURCE AT 90% PURITY)—<=1 GM (OVERAGE MAY BE ADDED TO ACCOUNT FOR DEGRADATION AND/OR PROCESSING LOSSES AND/OR SHELF LIFE); FENUGREEK SEED POWDER (FOR 1500 MG PER SERVING)—UPTO 3 GMS, TURMERIC POWDER (FOR 50 MG OF CURCUMINOIDS PER SERVING)—2.4 GMS; DHA OIL (FOR 200 MG PER SERVING)—0.5 GMS (OVERAGE MAY BE ADDED TO ACCOUNT FOR DEGRADATION AND/OR PROCESSING LOSSES AND/OR SHELF LIFE); VITAMIN D CHOLECALCIFEROL CONCENTRATE LIQUID (FOR 1000 IU PER SERVING)—1 MG (OVERAGE MAY BE ADDED TO ACCOUNT FOR DEGRADATION AND/OR PROCESSING LOSSES AND/OR SHELF LIFE); CALCIUM CITRATE (FOR 1000 MG PER SERVING)—2.35-4.7 GMS; NUT POWDER PER SERVING—UPTO 28 GMS, LGG (PER SERVING)—<=1 GM PURE CULTURE (OVERAGE MAY BE ADDED TO ACCOUNT FOR DEGRADATION AND/OR PROCESSING LOSSES AND/OR SHELF LIFE); FRUIT PUREE; YOGURT; WHEY PROTEIN CONCENTRATE; HYDROLYZED LACTOSE; ARTIFICIAL SWEETENERS; VITAMINS; MINERALS.

| | INGREDIENTS PER 8 OZ SERVING | |
|---|---|---|
| DAIRY NUT FRUIT BASED DRINK | AMOUNT | |
| YOGURT POWDER | 17 | GMS |
| NUT POWDER | 10 | GMS |
| DHA | 0.512 | GMS |
| HYDROLYZED LACTOSE | 8 | GMS |
| SAL (95% PURITY) | 7.7 | GMS |
| FENUGREEK POWDER | 3 | GMS |
| TURMERIC | 2.4 | GMS |
| VITAMIN D | 0.002 | GMS |
| CALCIUM CITRATE | 4.7 | GMS |
| OTHER VITAMINS | 0.1 | GMS |
| FLAVOR | 0.05 | GMS |
| OTHER (PROBIOTIC, STABILIZERS ETC) | 1 | GM |
| FRUIT PUREE | 15 | GMS |
| WATER | 175 | GMS |
| SERVING SIZE | 8 | OZ |

| | AMOUNT PER 100 GMS POWDER | |
|---|---|---|
| DAIRY FRUIT AND NUTS BASED POWDER | AMOUNT | |
| YOGURT POWDER | 24.47 | GMS |
| NUT POWDER | 14.40 | GMS |
| DHA | 0.74 | GMS |
| HYDROLYZED LACTOSE | 11.52 | GMS |
| SAL (95% PURITY) | 11.08 | GMS |
| FENUGREEK POWDER | 4.32 | GMS |
| TURMERIC | 3.46 | GMS |
| VITAMIN D | 2.88 | MG |
| CALCIUM CITRATE | 6.77 | GMS |
| OTHER VITAMINS | 0.14 | GMS |
| FLAVOR | 0.07 | GMS |
| OTHER (PROBIOTIC, STABILIZERS ETC) | 1.44 | GM |
| FRUIT PUREE | 21.59 | GMS |
| SUM | 100 | GMS |

### Example 4

In this example, a supplement for breastfeeding postpartum mothers provides sialyllactose, DHA, LGG, fenugreek, turmeric, calcium and Vitamin D.

### Example 4: Nut Based Product or Vegan: Powder, Refregerated ESL Liquid or Gummy or Pudding

Ingredients: SIALYLLACTOSE (FOR 10 GM PER SERVING)—(BOVINE SOURCE AT 4-4.2% SAL IN RAW MATERIAL)—240-300 GMS (OVERAGE MAY BE ADDED TO ACCOUNT FOR DEGRADATION AND/OR PROCESSING LOSSES AND/OR SHELF LIFE); SIALYLLACTOSE (FOR 10 GM PER SERVING)—(SYNTHETIC SOURCE AT 90% PURITY)—<=1 GM(OVERAGE MAY BE ADDED TO ACCOUNT FOR DEGRADATION AND/OR PROCESSING LOSSES AND/OR SHELF LIFE); FENUGREEK SEED POWDER (FOR 1500 MG PER SERVING)—UPTO 3 GMS; TURMERIC POWDER (FOR 50 MG OF CURCUMINOIDS PER SERVING)—2.4 GMS; DHA OIL (FOR 200 MG PER SERVING)—0.5 GMS; VITAMIN D CHOLECALCIFEROL CONCENTRATE LIQUID (FOR 1000 IU PER SERVING)—1 MG; CALCIUM CITRATE (FOR 1000 MG PER SERVING)—2.35-4.7 GMS; NUT MILK—6 OZ Or NUT POWDER PER SERVING—UPTO 28 GMS; LGG (PER SERVING)—<=1 GM PURE CULTURE (OVERAGE MAY BE ADDED TO ACCOUNT FOR DEGRADATION AND/OR PROCESSING LOSSES AND/OR SHELF LIFE); FRUIT PUREE; PLANT PROTEIN; HYDROLYZED LACTOSE; ARTIFICIAL SWEETENERS; VITAMINS; MINERALS.

| | INGREDIENTS PER 8 OZ SERVING | |
|---|---|---|
| NUT BASED VEGAN DRINK | AMOUNT | |
| NUT POWDER | 23 | GMS |
| DHA | 0.512 | GMS |
| HYDROLYZED LACTOSE | 8 | GMS |
| SAL (95% PURITY) | 7.7 | GMS |
| FENUGREEK POWDER | 3 | GMS |
| TURMERIC | 2.4 | GMS |
| VITAMIN D | 0.002 | GMS |
| CALCIUM CITRATE | 4.7 | GMS |
| OTHER (PROBIOTIC, STABILIZERS ETC) | 1 | GM |
| OTHER VITAMINS | 0.1 | GMS |
| FRUIT PUREE | 15 | GMS |
| PEA PROTEIN/PLANT PROTEIN POWDER | 3 | GMS |
| WATER | 175 | GMS |
| SERVING SIZE | 8 | OZ |

| NUT VEGAN FRUIT BASED POWDER | AMOUNT PER 100 GMS | |
|---|---|---|
| NUT POWDER | 33.62 | GMS |
| DHA | 0.75 | GMS |
| HYDROLYZED LACTOSE | 11.69 | GMS |
| SAL (95% PURITY) | 11.26 | GMS |
| FENUGREEK POWDER | 4.39 | GMS |
| TURMERIC | 3.51 | GMS |
| VITAMIN D | 2.92 | MG |
| CALCIUM CITRATE | 6.87 | GMS |
| OTHER (PROBIOTIC, STABILIZERS ETC) | 1.46 | GM |
| OTHER VITAMINS | 0.15 | GMS |
| FRUIT PUREE | 21.93 | GMS |
| PEA PROTEIN/PLANT PROTEIN | 4.39 | GMS |
| POWDER | | |
| SUM | 100 | GMS |

Formulation examples are provided to illustrate some embodiments of the nutritional composition of the present disclosure but should not be interpreted as any limitation thereon. Other embodiments within the scope of the claims herein will be apparent to one skilled in the art from the consideration of the specification or practice of the nutritional composition or methods disclosed herein. It is intended that the specification, together with all the examples disclosed herein, be considered to be exemplary only, with the scope and spirit of the disclosure being indicated by the claims, which follow the examples.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Although embodiments of the disclosure have been described using specific terms, devices, and methods, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of the various embodiments may be interchanged in whole or in part. Therefore, the spirit and scope of the appended claims should not be limited to the description of the versions contained therein.

1. A nutritional composition for supporting the health of a postpartum mother, comprising sialyllactose, a long chain polyunsaturated fatty acid (LCPUFA) and a probiotic.

2. The nutritional composition of claim 1, further comprising one or more ingredients selected from the group consisting of fenugreek, turmeric, nut extract, calcium, vitamin D and mixtures thereof.

3. The nutritional composition of claim 1 further comprising fenugreek, turmeric, and/or a nut extract.

4. The nutritional composition of claim 1, wherein the sialyllactose is present in an amount of about 7 to 10 g per serving of the nutritional composition.

5. The nutritional composition of claim 1, wherein the sialyllactose comprises 3′-Sialyllactose, 6′-Sialyllactose, or a combination of 3′-Sialyllactose and 6′-Sialyllactose.

6. The nutritional composition of claim 1, wherein the LCPUFA is present in an amount of about 150 mg to about 200 mg/serving of the nutritional composition.

7. The nutritional composition of claim 1, wherein the LCPUFA comprises docosahexaenoic acid (DHA).

8. The nutritional composition of claim 1, wherein the probiotic is present in an amount of up to about 1.5×10¹⁰ cfu per serving of the nutritional composition.

9. The nutritional composition of claim 1, wherein the probiotic comprises Lactobacillus rhamnosus GG (LGG).

10. The nutritional composition of claim 2, wherein the fenugreek is present in an amount of up to about 1500 mg per serving of the nutritional composition.

11. The nutritional composition of claim 2, wherein the turmeric is present in an amount sufficient to provide up to 50 mg total curcuminoids per serving of the nutritional composition.

12. The nutritional composition of claim 2, wherein the nut extract is present in an amount up to about 1.5 oz per serving of the nutritional composition.

13. The nutritional composition of claim 2, wherein the calcium is present in an amount of about 500 to about 1000 mg per serving of the nutritional composition.

14. The nutritional composition of claim 2, wherein the vitamin D is present in an amount of about 800 to about 1000 IU per serving of the nutritional composition.

15. The nutritional composition of claim 1, wherein the nutritional composition is in the form of a liquid or a powder.

16. The nutritional composition of claim 15, wherein the liquid nutritional composition is selected from the group consisting of a dairy-based drink, a fruit juice-based drink and a nut milk-based drink.

17. The nutritional composition of claim 16, wherein the dairy-based drink is a yogurt drink.

18. The nutritional composition of claim 15, wherein the liquid is packaged as a 6 to 7 oz serving and/or wherein the nutritional composition has 60-80 kcal/serving,

19. A method of supporting the health of a postpartum mother, the method comprising administering to the mother the nutritional composition of claim 1.

20. A method of preventing or reducing the severity of postpartum depression and/or anxiety in a postpartum mother, the method comprising administering to the mother the nutritional composition of claim 1.
